(19) United States
(12) Patent Application Publication
(10) Pub. No.: US 2020/0253510 A1
(43) Pub. Date: Aug. 13, 2020

(54) PROTECTIVE CLOTHING ARTICLE AND METHOD OF USE

(71) Applicant: Zanthion Inc., San Francisco, CA (US)

(72) Inventor: Philip Regenie, San Francisco, CA (US)

(73) Assignee: Zanthion Inc.

(21) Appl. No.: 16/787,854

(22) Filed: Feb. 11, 2020

Related U.S. Application Data
(60) Provisional application No. 62/803,851, filed on Feb. 11, 2019.

(51) Int. Cl. A61B 5/11; G08B 21/04

(52) U.S. Cl. CPC A61B 5/1117; G08B 21/043; A41D 1/06; A61B 2562/0219; G08B 21/0446

(57) ABSTRACT

A clothing protection system for protecting elderly people from falls and for monitoring data captured by the clothing protection system. The clothing protection system includes an article of clothing having at least one hidden pocket for holding a pad element. The at least one hidden pocket and the pad element are positioned adjacent to an impact point of the elderly person. The clothing protection system also includes at least one sensor supported by the article of clothing. The sensor can be positioned in close proximity to the pad element to monitor movement of the elderly person. A method of collecting and monitoring data related to falls of elderly people includes the step of implementing a clothing protection system for an elderly person. The method also includes the step of receiving fall data from the clothing protection system responsive to a fall for the elderly person.

### CROSS-REFERENCE TO RELATED APPLICATIONS

This application claims benefit under 35 U.S.C. 119(e) of U.S. Provisional Application Ser. No. 62/803,851, filed Feb. 11, 2019, which is hereby expressly incorporated herein by reference in its entirety.

### STATEMENT REGARDING FEDERALLY SPONSORED RESEARCH OR DEVELOPMENT

Not applicable.

### FIELD OF THE INVENTION

The present invention relates generally to a protective clothing system for protecting elderly people from falls and monitoring elderly people should falls occur. More specifically, the protective clothing system provides provide improved protection for the wearer and sensing of falls and activity for improved safety.

### BACKGROUND OF THE INVENTION

Falls are common among elderly members of our society. About one third of older people who live at home fall at least once a year, and about half of people who live in a nursing home fall. In the United States, falls are the leading cause of accidental death and the 7th leading cause of death in people age 65 or over. Once a person has had a fall, they are more likely to fall again.

Falls often cause injuries and some of the injuries, such as a broken hip, can be serious. Older people are more likely to break bones in falls because many older people have porous, fragile bones (osteoporosis), and some injuries caused by a fall are fatal.

Many older people fear falling. Fear of falling can lead to problems. People may worry about doing their usual activities and thus lose their self-confidence and even their independence. Older people can do many things to help overcome their fears and to reduce their risk of falling.

Accordingly, there is a need for a system to protect elderly people when they do fall and a system to monitor and record data in the event an elderly person does fall.

### SUMMARY OF THE INVENTION

The present disclosure is directed to a clothing protection system for protecting elderly people from falls and for monitoring data captured by the clothing protection system. The clothing protection system includes an article of clothing having at least one hidden pocket for holding a pad element. The at least one hidden pocket and the pad element are positioned adjacent to an impact point of the elderly person. The clothing protection system also includes at least one sensor supported by the article of clothing. The sensor can be positioned in close proximity to the pad element to monitor movement of the elderly person.

The present disclosure is also directed to a method of collecting and monitoring data related to falls of elderly people. The method includes the step of implementing a clothing protection system for an elderly person. The method also includes the step of receiving fall data from the clothing protection system responsive to a fall for the elderly person.

### BRIEF DESCRIPTION OF THE DRAWINGS

FIG. 1 is a front, side and rear elevation view of an article of clothing used in conjunction with a protecting clothing system in accordance with the present disclosure.

FIG. 2A is a frontal elevation view of an article of clothing incorporated in an embodiment of the protective clothing system in accordance with the present disclosure.

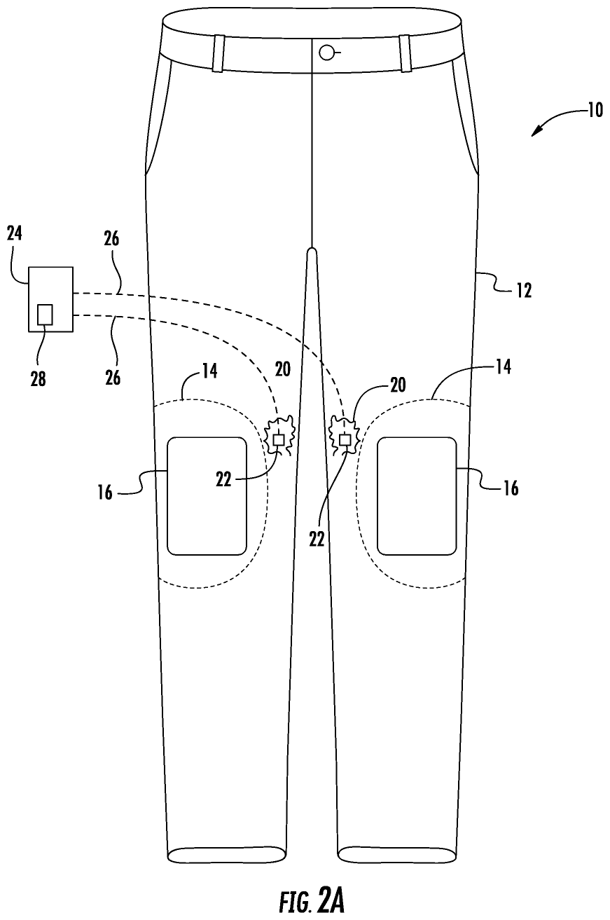

FIG. 2B is a back elevation view of the article of clothing shown in FIG. 2A in accordance with the present disclosure.

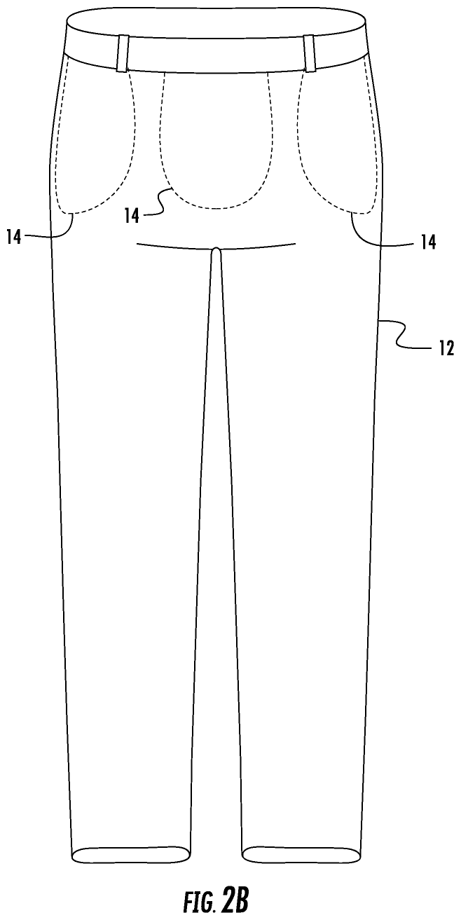

FIG. 2C is a side elevation view of the article of clothing shown in FIG. 2A in accordance with the present disclosure.

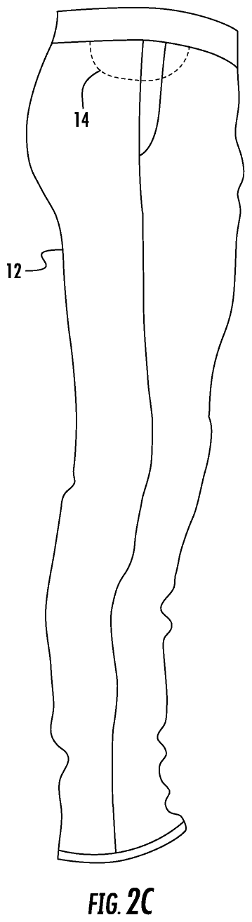

FIG. 3A is a perspective view of a portion of the article of clothing shown in FIGS. 2A-2C in accordance with the present disclosure.

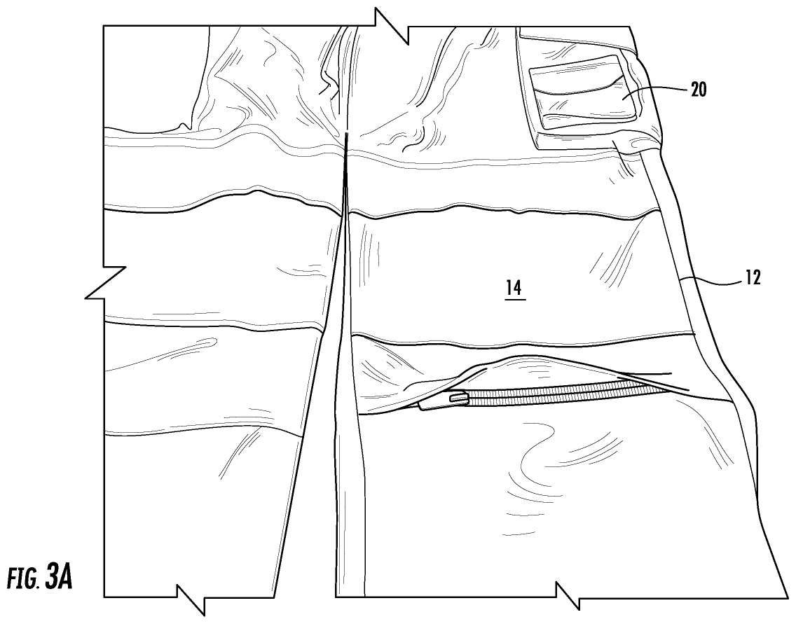

FIG. 3B is a perspective view of another portion of the article of clothing shown in FIGS. 2A-2C in accordance with the present disclosure.

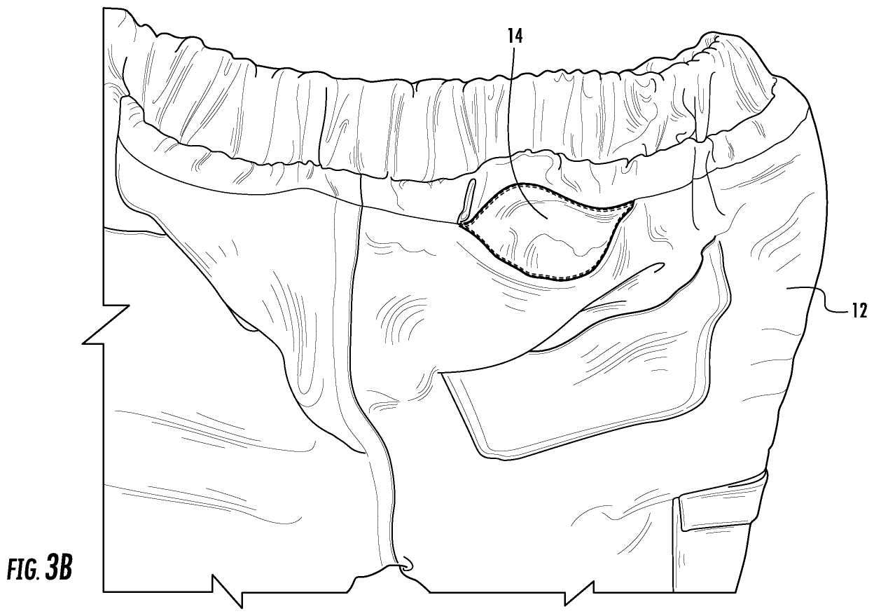

FIG. 3C is a perspective view of yet another portion of the article of clothing shown in FIGS. 2A-2C in accordance with the present disclosure.

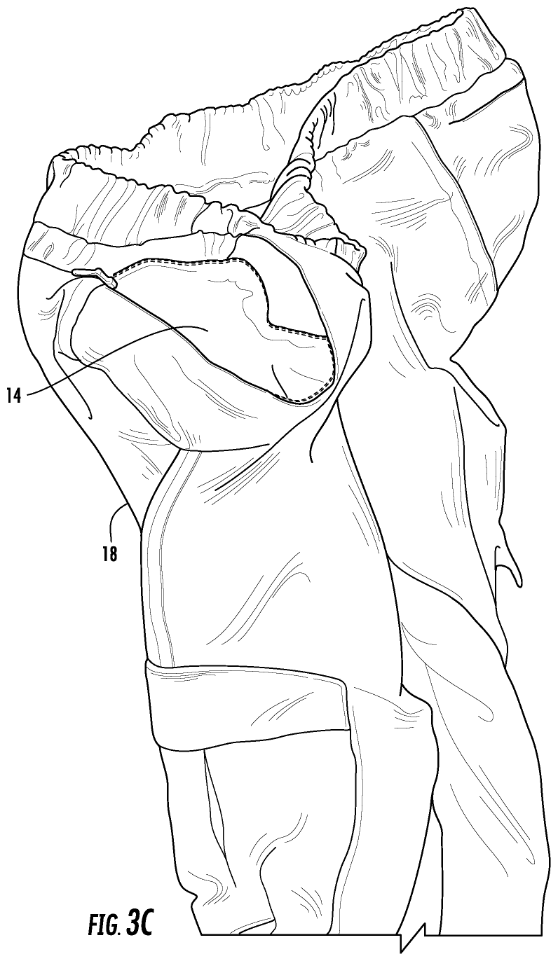

FIG. 4 is a perspective view of the portion of the article of clothing shown in FIG. 3C inserting a pad element in accordance with the present disclosure.

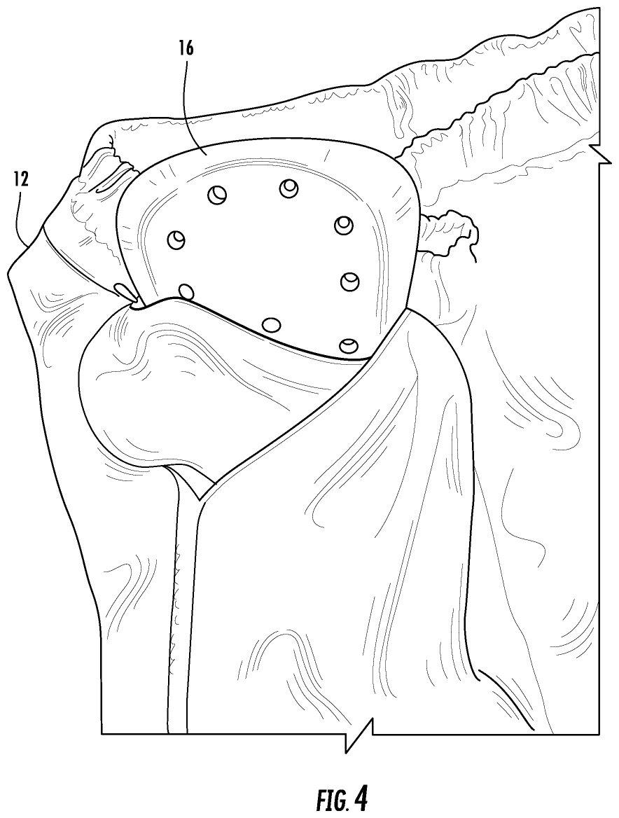

FIG. 5 is a perspective view of another portion of the article of clothing shown in FIGS. 2A-2C inserting a sensor in accordance with the present disclosure.

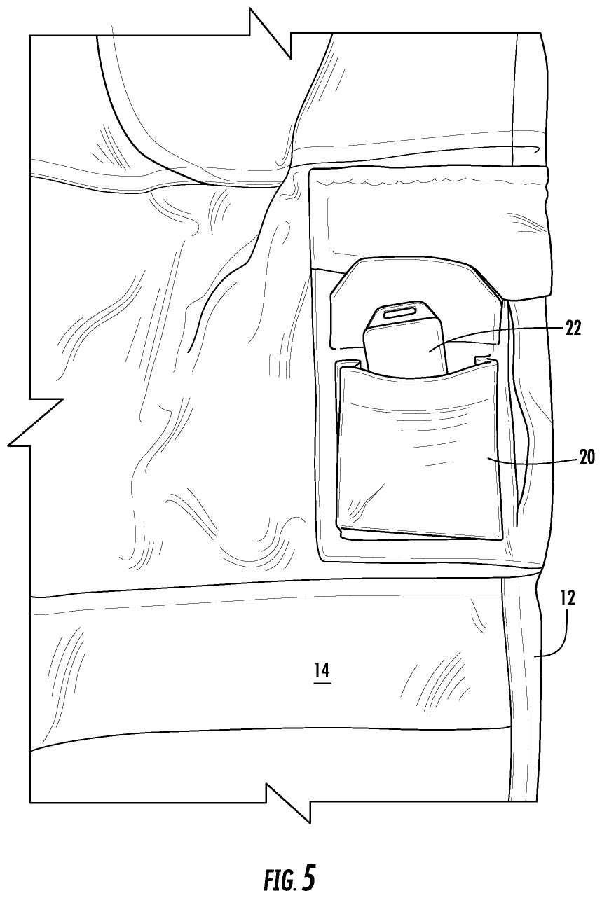

### DETAILED DESCRIPTION OF THE DISCLOSURE

The present disclosure is related to a clothing protection system 10 for protecting elderly people from injuries resulting from falls and for collecting and monitoring data captured by the clothing protection system 10. The data collected by the clothing protection system 10 can be used to improve the clothing protection system 10 and provide fall information to a database.

Referring now to the drawings, shown therein are various views and embodiments of the clothing protection system 10. In one embodiment, the clothing protection system 10 includes an article of clothing 12 having at least one hidden pocket 14 for housing a pad element 16 positioned at an impact point 18. The article of clothing 12 can be any article of clothing 12 that an elderly person may wear including, but not limited to, pants (as shown in the drawings), shorts, shirts, jackets or any other article of clothing. The hidden pockets 14 ensure the clothing article appears to be normal and makes the wearer feel good about wearing the clothing article, as there is no indication that the clothing is protective.

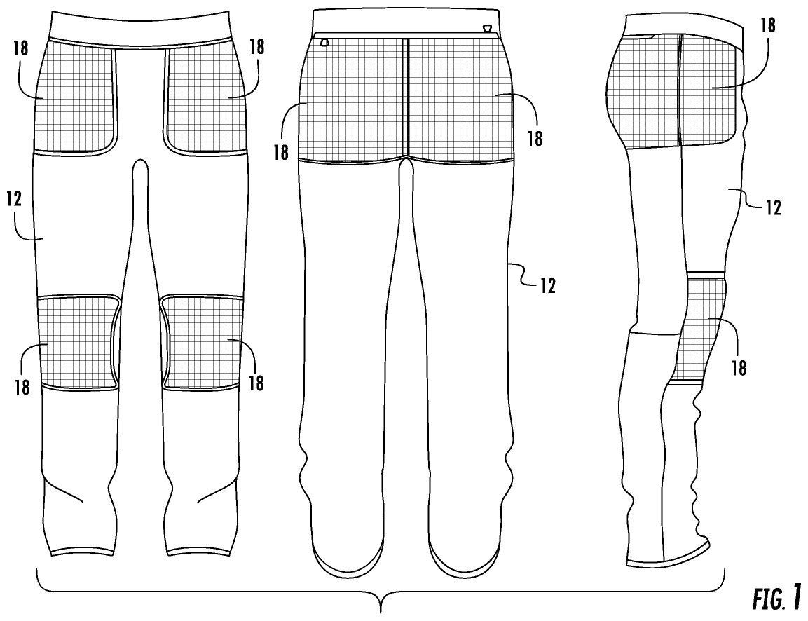

Common impact points 18 for the lower body are shown in FIG. 1. The common impact points 18 for a lower body of an elderly person include, but are not limited to, knees, hips, shoulders, coccyx, and the rear end. It should be understood and appreciated that other impact points 18 could be contemplated and accounted for in the clothing protection system 10. Not shown, but clearly contemplated by the present disclosure, is an article of clothing 12 for the upper body of an elderly person, such as a shirt, with impact points 18 at the elbows and shoulders, or any other location on the upper body where impact with the ground might occur during a fall.

In addition to the hidden pockets 14, the clothing protection system 10 can include at least one hidden enclosure 20 for containing at least one sensor 22 associated with a specific pad element 16 of the clothing protection system 10. For every pad element 16 the clothing protection system 10 includes, the clothing protection system 10 will include a sensor 22 associated with that pad element 16. In other words, if the clothing protection system 10 includes a pad element 16 in a hidden pocket 14 of the right knee, a sensor 22 will need to be positioned near the pad element 16 for the right knee.

Each sensor 22 can be positioned in close proximity to each pad element 16 to be able to capture data associated with movement of the elderly person at the location of each pad element 16. The sensor 22 is configured to detect activity and any user falls. In one embodiment, each sensor 22 is only separated from the associated pad element by layers of clothing material. In a further embodiment of the present disclosure, each sensor 22 can be supported by or attached to each associated pad element 16. For example, the pad element 16 can include a pocket for containing the associated sensor 22.

In one embodiment of the disclosure, the hidden pockets 14 can be easily accessible via known securing means such as a zipper, hook and loop closure, buttons, and the like. In another embodiment, the pad elements 16 can be secured directly to the clothing material or secured between two layers of clothing material without accessibility to the hidden pocket 14. Furthermore, the pad element 16 can be any type of material capable of minimizing the impact on an elderly person from a fall. The pad element 16 can be constructed of polymeric materials such as a carbon fiber lattice that distributes the impact across a wider part of the pad element 16 than only where the exact impact occurred.

The sensors 22 of the clothing protection system 10 can be any sensor known in the art capable of tracking and recording movement. In one embodiment, the sensor is an accelerometer that can track movement and determine impact data for each impact point 18 from the vibration of the impact point's associated accelerometer. The data from each accelerometer can also provide information about when each specific impact point 18 made impact. For example, the clothing protection system 10 can determine via the accelerometer data that an elderly person fell and impacted their right knee and then impacted their right hip and elbow. From the accelerometer data and other data, such as height and weight of the elderly person, the pad elements 16 can be designed specifically for each elderly person.

The clothing protection system 10 can also include a computing device 24 that can be in wireless communication via a wireless communication link 26 with the sensors 22. The computing device 24 can include an activity and fall detection system 28 monitor the data from the sensors 22 to determine if a fall has occurred. The activity and fall detection system 28 of the computing device 24 can notify various entities, such as family members, health care professionals, emergency responders, and the like, in the event a fall has occurred. The activity and fall detection system 28 of the computing device 26 can record the data detected by the sensors 22 to determine forces associated on the various impact points 18 to generate a database. The computing devices 24 can be any device capable of capturing the data from the sensors 22, such as a cell phone, computer, and the like. The computing devices 24 would need a memory for storing the data and programming information for a central processing unit of the computing devices 24. The computing devices 24 can also include a graphical user interface to allow a user to operate the computing devices 24. The computing devices 24 can also include any other hardware or software needed to operate the clothing protection system 10 as described herein.

The present disclosure is also directed to a method of using the clothing protection system 10 to monitor capture fall data associated with the activity and falls of elderly individuals. The clothing protection system 10 can capture data form the sensors 22 related to movement and activity of the clothing protection system 10 user. The clothing protection system 10 can receive data from the sensors indicative of a fall. The clothing protection system 10 via the computing devices 24 can send an emergency alert to various entities depending on the data from the sensors. If the fall was of a certain intensity or activity after the fall condition is limited to a certain amount, calls or messages can be sent to the proper entity. The data from the sensors 22 can be stored on the computing devices 24 for analysis and optimization. Additional data can be generated from the data received from the sensors 22. The fall data can be analyzed to generate impact data associated with the fall. The impact data can then be used to design a specific pad elements for the various impact points for a particular user.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. A clothing protection system for protecting elderly people from falls and for monitoring data captured by the clothing protection system, the clothing protection system comprising:
an article of clothing having at least one hidden pocket for holding a pad element, the at least one hidden pocket and the pad element are positioned adjacent to an impact point of the elderly person; and
at least one sensor supported by the article of clothing positioned in close proximity to the pad element to monitor movement of the elderly person.

2. The system of claim 1 wherein the article of clothing further includes at least one hidden enclosure for containing the at least one sensor.

3. The system of claim 1 wherein the article of clothing has a plurality of hidden pockets and at least one sensor associated with each hidden pocket to monitor movement of the elderly person.

4. The system of claim 1 wherein the at least one sensor is an accelerometer.

5. The system of claim 1 wherein the at least one sensor communicates with a computing device.

6. The system of claim 5 wherein the computing device receives and stores information received from the at least one sensor.

7. The system of claim 6 wherein the computing device generates impact data from information received from the at least one sensor for impact points of the elderly person after a fall has occurred.

8. The system of claim 1 wherein the at least one sensor is supported by the pad element of the clothing protection system.

9. The system of claim 1 wherein the at least one hidden pocket is selectively accessible to permit the pad element to be taken in and out or the at least one hidden pocket can be inaccessible wherein the pad element is secured between layers of the article of clothing.

10. A method of collecting and monitoring data related to falls of elderly people, the method comprising:
implementing a clothing protection system for an elderly person; and
receiving fall data from the clothing protection system responsive to a fall for the elderly person.

11. The method of claim 10 further comprising the step of storing the data received from the clothing protection system.

12. The method of claim 10 further comprising the step of generating impact data from the fall data received from the clothing protection system.

13. The method of claim 12 further comprising the step of designing a clothing protection system based on the impact data generated from a fall.

14. The method of claim 10 wherein the clothing protection system can send an emergency alert to various entities when it has been determined that an elderly person has fallen.

15. The method of claim 10 wherein the clothing protection system comprises:
an article of clothing having at least one hidden pocket for holding a pad element, the at least one hidden pocket and the pad element are positioned adjacent to an impact point of the elderly person; and
at least one sensor supported by the article of clothing positioned in close proximity to the pad element to monitor movement of the elderly person.

16. The method of claim 15 wherein the article of clothing further includes at least one hidden enclosure for containing the at least one sensor.

17. The method of claim 15 wherein the article of clothing has a plurality of hidden pockets and at least one sensor associated with each hidden pocket to monitor movement of the elderly person.

18. The system of claim 15 wherein the at least one sensor communicates with a computing device.

19. The system of claim 15 wherein the at least one sensor is supported by the pad element of the clothing protection system.

20. The system of claim 15 wherein the at least one hidden pocket is selectively accessible to permit the pad element to be taken in and out or the at least one hidden pocket can be inaccessible wherein the pad element is secured between layers of the article of clothing.
